(19) United States
(12) Patent Application Publication
(10) Pub. No.: US 2017/0150886 A1
(43) Pub. Date: Jun. 1, 2017

(54) FORCE DETECTING APPARATUS

(71) Applicant: CHIA-WEI LIN, TAINAN CITY (TW)

(72) Inventors: CHIA-WEI LIN, TAINAN CITY (TW); CHI-JU LEE, KAOHSIUNG CITY (TW); WEI-CHENG LAI, TAIPEI CITY (TW)

(21) Appl. No.: 15/360,841

(22) Filed: Nov. 23, 2016

Related U.S. Application Data
(60) Provisional application No. 62/261,168, filed on Nov. 30, 2015.

(51) Int. Cl. A61B 5/00

(52) U.S. Cl. CPC A61B 5/0053

(57) ABSTRACT

A force detecting apparatus includes a button and a force sensor. The button is configured to press a biological tissue. The force sensor is disposed between the button and the biological tissue for detecting a force variation between the button and the biological tissue.

### CROSS-REFERENCE TO RELATED APPLICATIONS

This application claims the benefit of U.S. Provisional Application No. 62/261,168, filed Nov. 30, 2015, the entire disclosure of which is incorporated by reference herein.

### FIELD

The disclosure relates to a detecting apparatus, more particular to a force detecting apparatus.

### BACKGROUND

The rotator cuff is a group of muscles at an innermost layer of the shoulder joint, including supraspinatus, infraspinatus, teres minor and subscapularis, is a core muscle group of the shoulder joint, and has a main function of stabilizing humerus when the shoulder joint moves. When suffering from a trauma or being repetitively used, for example, suffering from collision or continuously lifting an arm to clean a high place, the rotator cuff may be worn, or even suffer from fibrosis and tear. Moreover, aging is also one important pathogenic factor. According to statistics, approximately 13% of the population at an age greater than 50 suffers from rotator cuff tendon tear, and over half of the population at an age greater than 80 is diagnosed with a rotator cuff tendon injury. After the rotator cuff injury, possible clinical manifestations include: being painful (sleep may be affected), being incapable of completing an action over the shoulder, being difficult in getting dressed and other disorders of repetitive shoulder function movements.

The extent of the rotator cuff tendon injury or tear may be divided into three stages, and symptoms and therapies of the rotator cuff tendon injury or tear are shown in Table 1.

TABLE 1

| Grade | Symptom | Therapy |
|---|---|---|
| I | edema nearby the muscle tendon, and | physical therapy and drug control |
| | bleeding (usually at an age less than 25) | |
| II | tendinitis/bursitis and fibrosis (usually at an | physical therapy and drug |
| | age of 25 to 40) | control, or taking surgery |
| | | intervention into consideration in |
| | | case of severity |
| III | bone spur and muscle tendon tear (usually at | putting stress on surgery |
| | an age greater than 40) | intervention |

In Table 1, the part of the surgery intervention therapies is further roughly classified into a conventional open type, a mini open type and an arthroscopic method, as shown in Table 2.

TABLE 2

| | Conventional open | | |
|---|---|---|---|
| | type | Mini open type | Arthroscopy |
| shoulder joint | x | ∘ | ∘ |
| assessment | | | |
| wound size | 4 to 6 cm | 3 to 4 cm | 0.4 to 0.7 cm |
| deltoid invasion extent | excision and repair | pushing aside | tiny |
| repair manner | bone tunnel | bone tunnel fixation | single-row or |
| | fixation | or single-row suture | double-row suture |
| | | anchor | anchor, and bone |
| | | | tunnel fixation |
| postoperative initial | ∘ | ∘ | ∘ |
| passive movement | | | |
| postoperative initial | x | ∘ | ∘ |
| active assisted | | | |
| movement | | | |
| postoperative initial | x | ∘ | ∘ |
| active movement | | | |
| postoperative pain | moderate to severe | moderate | mild |
| rehabilitation period | 9 to 12 months | 4 to 12 months | 3 to 6 months |

Because the wound is small, and the postoperative recovery is quick, more orthopedists tend to perform suture anchor by using the arthroscopy to assist in fixing the muscle tendon to a bone, and this surgical manner is slowly used as a standard process of rotator cuff repair. However, the suture anchor is still incapable of completely fixing the rotator cuff, and a case of failure still occurs, for example, a fixing screw is pulled out due to osteoporosis, or the rotator cuff tendon is torn again because of a suture. Regretfully, the circumstances mentioned above cannot be predicted in advance. If the fixing situation of the rotator cuff can be monitored in real time, a preventive action can be done before the circumstances mentioned above happened.

### SUMMARY OF THE INVENTION

In accordance with one aspect of the present disclosure, a force detecting apparatus includes a button and a force sensor. The button is configured to press a biological tissue. The force sensor is disposed between the button and the biological tissue for detecting a force variation between the button and the biological tissue.

In the present disclosure, the button can press the biological tissue, and to the force sensor can detect the force variation between the button and the biological tissue in real time. Accordingly, the force detecting apparatus is suitable for monitoring the mechanical behaviors of the biological tissue or reattaching the biological tissue to a hard tissue.

### BRIEF DESCRIPTION OF THE DRAWINGS

Aspects of the present disclosure are understood from the following detailed description when read with the accompanying figures. It is emphasized that, in accordance with the standard practice in the industry, various features are not drawn to scale. In fact, the dimensions of the various features may be arbitrarily increased or reduced for clarity of discussion.

FIG. 1 is a schematic view of a force detecting apparatus in accordance with some embodiments of the present disclosure.

FIG. 2 illustrates a schematic view of a force detecting apparatus for reattaching a biological tissue to a hard tissue in accordance with some embodiments of the present disclosure.

FIG. 3 is a side view of a button contacting with a biological tissue in accordance with some embodiments of the present disclosure.

FIG. 4 illustrates a schematic view of a force detecting apparatus for reattaching a biological tissue to a hard tissue in accordance with some embodiments of the present disclosure.

FIG. 5 is a schematic view of a force sensor in accordance with some embodiments of the present disclosure.

### DETAILED DESCRIPTION OF THE INVENTION

It is to be understood that the following disclosure provides many different embodiments or examples, for implementing different features of various embodiments. Specific examples of components and arrangements are described below to simplify the present disclosure. The present disclosure may, however, be embodied in many different forms and should not be construed as being limited to the embodiments set forth herein; rather, these embodiments are provided so that this description will be thorough and complete, and will fully convey the present disclosure to those of ordinary skill in the art. It will be apparent, however, that one or more embodiments may be practiced without these specific details.

In addition, the present disclosure may repeat reference numerals and/or letters in the various examples. This repetition is for the purpose of simplicity and clarity and does not in itself dictate a relationship between the various embodiments and/or configurations discussed.

It will be understood that singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms; such as those defined in commonly used dictionaries, should be interpreted as having a meaning that is consistent with their meaning in the context of the relevant art and the present disclosure, and will not be interpreted in an idealized or overly formal sense unless expressly so defined herein.

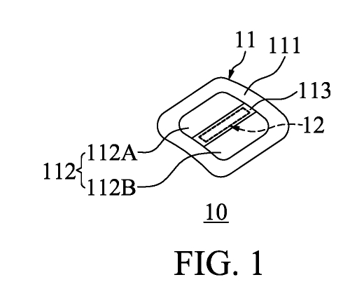

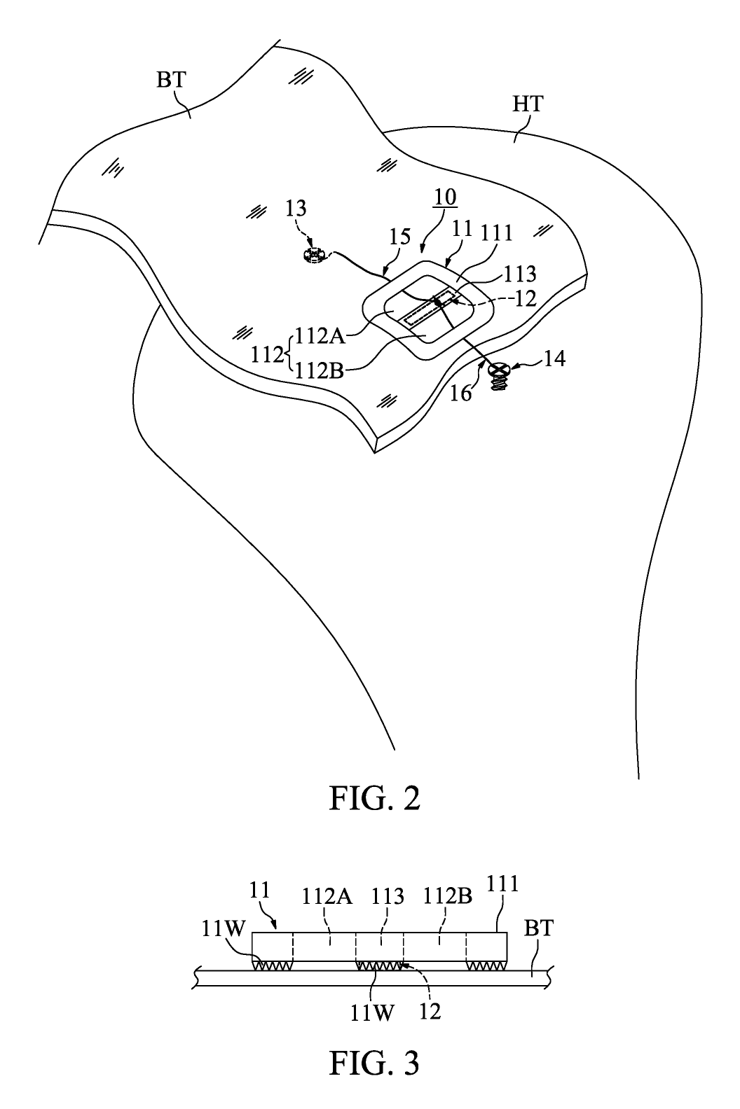

Referring to FIGS. 1 and 2, a force detecting apparatus 10 is designed to press a biological tissue BT. In some embodiments, the biological tissue BT is a soft tissue such as rotator cuff. In some embodiments, the biological tissue BT can also be a hard tissue such as bone chip. In some embodiments, the force detecting apparatus 10 is used to reattach the biological tissue BT (as rotator cuff) to a hard tissue HT (as humerus).

The force detecting apparatus 10 includes a button 11 configured to press the biological tissue BT on the hard tissue HT. The button 11 includes a frame portion 111, a hollow portion 112 and a bisecting rod 113. The hollow portion 112 is located at the middle of the frame portion 111. The bisecting rod 113 is disposed within the hollow portion 112 for dividing the hollow portion 112 into a first hollow portion 112A and a second hollow portion 112B. The bisecting rod 113 is also located at the middle of the frame portion 111 and two ends of the bisecting rod 113 are connected to the frame portion 111. In some embodiments, the button 11 is made of bioabsorbable material. In some embodiments, the bisecting rod 113 can be omitted, it only needs two holes and the frame portion 111 and the bisecting rod 113 are in the same part.

Referring to FIG. 3, in order to improve the contact force between the button 11 and the biological tissue BT, the button 11 can include a sawtooth structure 11W contacting with the biological tissue BT to prevent sliding between the button 11 and the biological tissue BT. In some embodiments, the sawtooth structure 11W is formed on the frame portion 111. In some embodiments, the sawtooth structure 11W is formed on the bisecting rod 113. In some embodiments, the frame portion 111 can integrate with soft or elastic material to prevent biological tissue attrition caused by the friction between the button 11 and the biological tissue BT.

Referring to FIGS. 1 and 2 again, a force sensor 12 is disposed between the button 11 and the biological tissue BT for detecting a force variation between the button 11 and the biological tissue BT. To improve the accuracy of the detected force variation, the force sensor 12 is disposed between the bisecting rod 113 and the biological tissue BT. In some embodiments, the force sensor 12 is disposed on the button 11, preferably, the force sensor 12 is directly disposed or formed on the bisecting rod 113 of the button 11. In some embodiments, the bisecting rod 113 can have a cavity to dispose the force sensor 12. In some embodiments, the force sensor 12 can be fixed by a suture. In some embodiments, the force detecting apparatus 10 can receive a wireless power.

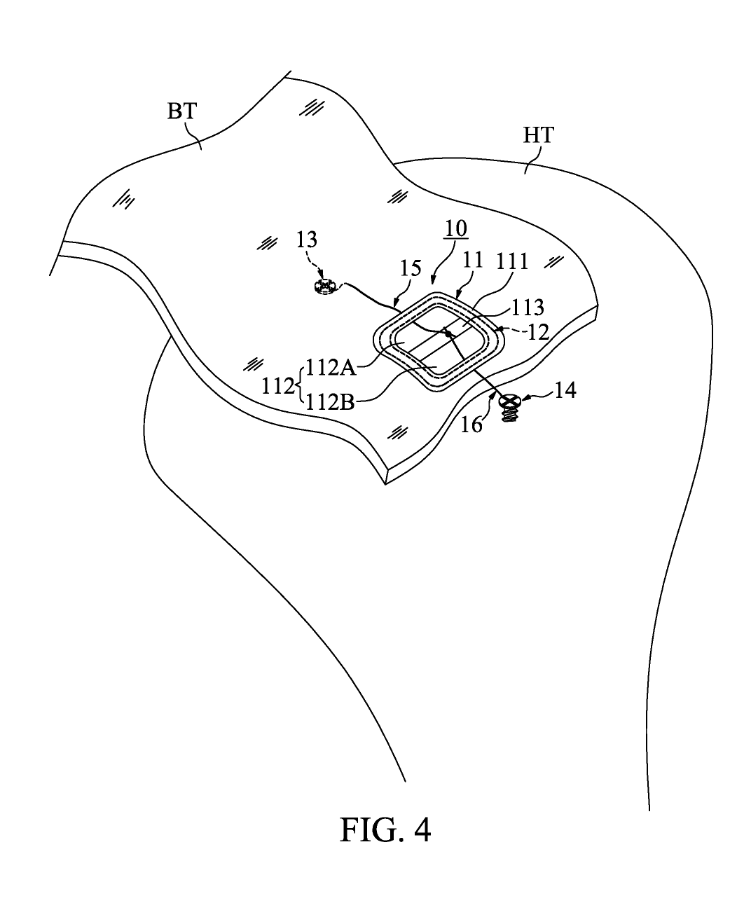

Referring to FIG. 4, in some embodiments, the force sensor 12 can be disposed between the frame portion 111 and the biological tissue BT. In some embodiments, the force sensor 12 is directly disposed or formed on the frame portion 111 of the button 11.

The force sensor 12 is selected from a group consisting of pressure sensor, shear force sensor and tensile force sensor. Accordingly, the force variation can be pressure variation, shear force variation or tensile force variation.

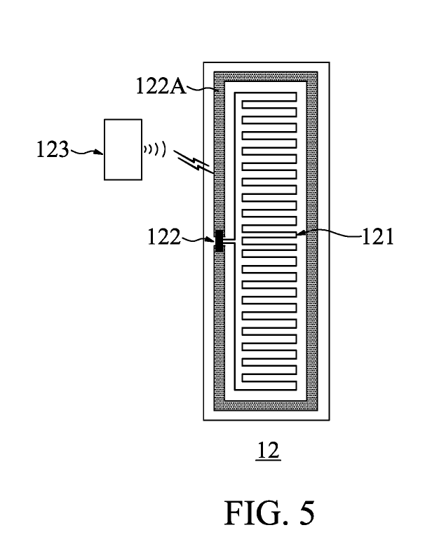

Referring to FIGS. 1 and 5, the force sensor 12 includes a sensing element 121 for detecting the force variation and a transponder 122 for transmitting the force variation signal to a signal receiver 123. The sensing element 121 is electrically connected to the transponder 122. The sensing element 121 is made of one selected from the group consisting of piezoresistive material, piezoelectric material, capacitive material and resistance material. In some embodiments, the transponder 122 is radio frequency identification (RFID) tag, and the signal receiver 123 is radio frequency identification (RFID) reader. In some embodiments, the transponder 122 has an antenna 122A to transmit the force variation signal. Preferably, the antenna 122A is made of bioabsorbable material. The antenna 122A can be also used to receive the wireless power that the force detecting apparatus 10 can with or without battery to save the area. In some embodiments, the antenna 122A can be disposed on the frame portion 111. In some embodiments, the force sensor 12 and the transponder 122 can be separated. The transponser 122 can be in/on the button 11. In some embodiments, the wireless data communication between the transponder 122 and the signal receiver 123 can use bluetooth or WiFi.

Referring to FIG. 2 again, in order to generate a pressure on the button 11 to uniformly press the biological tissue BT on the hard tissue HT, the force detecting apparatus 10 can include a first suture anchor 13 and a second suture anchor 14. The first suture anchor 13 and the second suture anchor 14 are separately disposed at two sides of the button 11. In some embodiments, the first suture anchor 13 and the second suture anchor 14 are fixed on the hard tissue HT.

A first suture 15 is secured to the first suture anchor 13 and penetrates the biological tissue BT and the first hollow portion 112A of the button 11.

A second suture 16 is secured to the second suture anchor 14 and penetrates the second hollow portion 112B of the button 11. The first suture 15 and the second suture 16 are knotted on the bisecting rod 113 of the button 11, thereby generating a pressure on the button 11 to uniformly press the biological tissue BT on the hard tissue HT.

In the present disclosure, the button 11 can press the biological tissue BT, and the force sensor 12 can detect the force variation between the button 11 and the biological tissue BT in real time and can transmit the force variation signal in wireless. Accordingly, the force detecting apparatus 10 is suitable for monitoring the mechanical behaviors of the biological tissue BT or reattaching the biological tissue BT to a hard tissue HT.

Moreover, the scope of the present application is not intended to be limited to the particular embodiments of the process, machine, manufacture, and composition of matter, means, methods and steps described in the specification. As those skilled in the art will readily appreciate form the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure.

Accordingly, the appended claims are intended to include within their scope such processes, machines, manufacture, and compositions of matter, means, methods or steps. In addition, each claim constitutes a separate embodiment, and the combination of various claims and embodiments are within the scope of the invention.

What is claimed is:

1. A force detecting apparatus, comprising:
a button; and
a force sensor disposed on the button.

2. The force detecting apparatus of claim 1, wherein the button is configured to press a biological tissue, and the force sensor is disposed between the button and the biological tissue for detecting a force variation between the button and the biological tissue.

3. The force detecting apparatus of claim 2, wherein the button comprises a hollow portion and a bisecting rod, the bisecting rod is disposed within the hollow portion for dividing the hollow portion into a first hollow portion and a second hollow portion, and the force sensor is disposed between the bisecting rod and the biological tissue.

4. The force detecting apparatus of claim 3, further comprising a first suture anchor, a second suture anchor, a first suture and a second suture, wherein the first and second suture anchors are separately disposed at two sides of the button, the first suture is secured to the first suture anchor and penetrates the first hollow portion of the button, and the second suture is secured to the second suture anchor and penetrates the second hollow portion of the button.

5. The force detecting apparatus of claim 4, wherein the first suture and the second suture are knotted on the bisecting rod of the button.

6. The force detecting apparatus of claim 4, wherein the first and second suture anchors are fixed on a hard tissue.

7. The force detecting apparatus of claim 2, wherein the button comprises a frame portion and a bisecting rod, the bisecting rod is located at the middle of the frame portion and two ends of the bisecting rod are connected to the frame portion, and the force sensor is disposed between the frame portion and the biological tissue.

8. The force detecting apparatus of claim 2, wherein the button comprises a sawtooth structure contacting with the biological tissue to prevent sliding between the button and the biological tissue.

9. The force detecting apparatus of claim 8, wherein the button comprises a frame portion and a bisecting rod, the bisecting rod is located at the middle of the frame portion and two ends of the bisecting rod are connected to the frame portion, and the sawtooth structure is formed on the frame portion.

10. The force detecting apparatus of claim 8, wherein the button comprises a frame portion and a bisecting rod, the bisecting rod is located at the middle of the frame portion and two ends of the bisecting rod are connected to the frame portion, and the sawtooth structure is formed on the bisecting rod.

11. The force detecting apparatus of claim 1, wherein the force sensor is selected from a group consisting of pressure sensor, shear force sensor and tensile force sensor.

12. The force detecting apparatus of claim 2, wherein the force sensor comprises a sensing element for detecting the force variation and a transponder for transmitting the force variation signal to a signal receiver.

13. The force detecting apparatus of claim 12, wherein the transponder is radio frequency identification (RFID) tag, and the signal receiver is radio frequency identification (RFID) reader.

14. The force detecting apparatus of claim 2, wherein the force variation is pressure variation, shear force variation or tensile force variation.

15. The force detecting apparatus of claim 1, wherein the button comprises a bisecting rod, the force sensor is disposed on the bisecting rod.

16. The force detecting apparatus of claim 1, wherein the button comprises a frame portion, the force sensor is disposed on the frame portion.
